(19) United States
(12) Patent Application Publication
(10) Pub. No.: US 2022/0362116 A1
(43) Pub. Date: Nov. 17, 2022

(54) ANTIMICROBIAL SKIN CREAM

(71) Applicant: Yvon GANGA, San Diego, CA (US)

(72) Inventor: Yvon GANGA, San Diego, CA (US)

(21) Appl. No.: 17/624,136

(22) PCT Filed: Jul. 3, 2019
(86) PCT No.: PCT/US2019/040576

§ 371 Date: Dec. 30, 2021

(51) Int. Cl. A61K 8/25; A61K 8/92; A61K 8/31; A61K 8/368; A61Q 19/00; A61Q 17/00

(52) U.S. Cl. CPC A61K 8/25; A61K 8/925; A61K 8/31; A61K 8/368; A61Q 19/007; A61Q 17/005; A61K 2800/75

(57) ABSTRACT

This invention relates to compounds that treat microbial skin infections. Previously, powered forms of talk were difficult to apply to skin without becoming a lung irritant. Embodiments of the present invention use talc, lanolin, petroleum jelly, sodium lauryl ether sulfate, sodium chloride, water, sodium salicylate, Lauryl ether sulfate, Isopropyl palmitate, propylene glycol, mineral oil, and sodium benzoate.

### TECHNICAL FIELD

The embodiments herein relate generally to skin care, and more particularly, to an antimicrobial skin cream.

### BACKGROUND ART

Young children are prone to inhaling the baby powder that is used on their skin, especially if it has been made from talc. Talcum powder has very fine particles which can be inhaled and cause irritation to the baby's delicate lungs. One will have to be especially careful if one's baby has any kind of respiratory illness like asthma since, with baby talcum powder, some danger may be possible. Also, Talcum dusting powder is commonly used to reduce rashes and diaper irritation in babies and infants. But this practice is dangerous. It can result in the inhalation of significant amounts of powder, causing acute or chronic lung irritation, known as talcosis.

Therefore, what is need is an antimicrobial baby cream that helps prevent and protect a baby's skin from dryness.

### DISCLOSURE OF THE INVENTION

Some embodiments of the present disclosure include a cream for treating the skin. The cream may include talc, lanolin, petroleum jelly, sodium lauryl ether sulfate, sodium chloride, water, sodium salicylate, Lauryl ether sulfate, Isopropyl palmitate, propylene glycol, mineral oil, and sodium benzoate. The cream may also include ortho-silicic acid powder, glycerin, gelatin, vitamin A, vitamin E, vitamin D, vitamin C, vitamin B2, vitamin B5, vitamin H, vitamin B6, and vitamin D.

### BRIEF DESCRIPTION OF THE FIGURES

The detailed description of some embodiments of the invention is made below with reference to the accompanying figures, wherein like numerals represent corresponding parts of the figures.

FIG. 1 shows a graph of a theory related to the princeps of the invention;

FIG. 2 shows a graph of a theory related to the princeps of the invention;

FIG. 3 shows a graph of a theory related to the princeps of the invention;

FIG. 4 shows a graph of a theory related to the princeps of the invention;

FIG. 5 shows a graph of a theory related to the princeps of the invention; and

FIG. 6 shows a graph of a theory related to the princeps of the invention.

### BEST MODE OF THE INVENTION

For more precision, all graphs are the mathematical device for representing relationship reactivities and co-reactivities between the fundamental thermodynamic force and microcrack extension stress stability (Entropy) on the Antimicrobial cream (Princeps), displaying instability and stability phase reactions. The graphs display the principle of manifestations of two phase reactions in the universe, Antimicrobial skin cream(Princeps) as well in the present invention (Antimicrobial Baby cream)

The Antimicrobial baby cream Invention is emanated or generated from Mirror virtues Antimicrobial cream's reactivities and active ingredient (Ortho-silicic acid) function. The Antimicrobial cream final formula(Pinceps) is: (Al,Zn,Fe₁,67MgO₃₃)Si₄O₁₀(OH)₂Na⁺Ca⁺⁺)

The Galenic structure is: Mg₃Si₄O₁₀(OH)₂(s)+Al₂Si₂O₅(OH)₄(s)+15H₂O—→6(H₄SiO₄)(s)+2Al(0H)₃+3Mg(OH)₂

We have the formation of Ortho-silicic acid. During this reaction, the formation of ortho-silicic acid, aluminum hydroxide and magnesium hydroxide may occur, provided by the complete hydrolysis of Kaolin (Al2Si2O5(OH)4) and talc activated by the amount of sodium chloride, Lauryl ether sulfate, and fatty acids. In the presence of Blue Tartrazine, and vitamins. The Ortho-silicic acid active ingredient's reactivity serves reference to produce the Baby Antimicrobial cream we have. Obviously, the chemical potential of the Antimicrobial skin cream and physical potential influence and control the reactivities formation of Antimicrobial Baby cream under the influence of the fundamental thermodynamic force law. The Antimicrobial baby skin may be synthetized with two reactions. The ortho-silicic and Magnesium Hydroxide cream acid by talc hydrolyze in the presence of vitamin. We have:

2Mg₃SiO₄(OH)₂+6H₂O→SiO₂₊H₄SiO₄₊₆Mg (OH)2 PH=7.4

We may also, synthetize the ortho-silicic and Magnesium hydroxide by hydrolyze Talc in the presence of small amount of ortho-silicic acid we have:

2Mg₃SiO₄(OH)₂+H₄SiO₄+6H₂O→SiO₂+2H₄SiO₄+6Mg(OH)₂ PH=7.4

Physicochemical potential of Antimicrobial cream and the fundamental thermodynamic force of the universe guides, influence, and control the reactivities of the Antimicrobial Baby cream. In a complex environment, the chemical potential of species influences and have an impact for the effect of the rest of the environment on the species in question (especially the Antimicrobial skin cream also containing water and ortho-silicic acid). The fundamental thermodynamic force and its legitimate function exert an impact on the chemical potential and physical potential of all entities and living system in the universe on the quest to favor all universal reaction and interaction.

The chemical potential of the Antimicrobial skin cream allows in particular the study of the phenomenon of diffusion of the material, open or flux induction Thermodynamic systems, by translation of the mirrors effects, the reaction systems and the equilibrium conditions of its chemical reactions and phase balance. This analogy and mirror effect by translation is a great privilege to conciliate and value the therapeutics effect of the Antimicrobial skin cream. The chemical potential of Antimicrobial cream has the enormous advantage of being a fundamental quantity, as well as temperature, deduced from the principles of thermodynamics and thus benefit from the physical and mathematical arsenal of this discipline. The Antimicrobial skin cream overflows mineral fundamental elements. Technology also includes all branches of science such as mathematics, physics, chemistry, topology, The mechanics of fluids, Mechanics, electricity, and the electrostatic potential. In addition, the therapeutic abilities and healing method Antimicrobial skin cream with the formation of Ortho- silicic relates as ingredient active relates rationally and evidence the relation between the fundamental thermodynamic force and human Entropy physiology. The virtues of this Antimicrobial skin technology by translation or mirror effects designates or identify Ortho-silicic acid reactivity as reference to produce cream and explains the thermodynamic reactivity between inorganic colloids and metalloproteins (cations and anions), electrics charge reactivities during osmotic and crystalline swelling and final phase. Obviously, osmotic and crystalline swelling refers to instability process or the Matrix thermodynamic phase (on the search or on the quest of thermodynamic stability), and the Quantum Metrix Antigravity in terminal synthesis phase (establishment of strict stability). Moreover, the fundamental thermodynamic process enables to favor chemical reactions in case of several phases to reach equilibrium state. When the chemical system in several phases can coexist, each constituent will transfer from the phase in which its chemical potential is highest to the phase in which its chemical potential is the weakest, until these chemical potentials equalize. The Antimicrobial skin cream with the formation of ortho-silicic acid prefers directly related to the experiment: capillary pressure, equilibrium relative humidity in the case of hygroscopic media. The physicochemical potential testifies to the evolution and the situation of the thermodynamic stability of the Antimicrobial cream during the synthesis phase. The thermodynamic stability during the synthesis process of Antimicrobial cream evolves and varies in two phases. The first phase (Matrix thermodynamic stability) is a phase of instability in search of thermodynamic stability during crystalline and osmotic swelling, so this phase generates topological deformations with presence of curvature (Normal, regular multiform, irregular multiform, Reimann, Boltzmann, Ricci curvature) in search of stability it proceeds kinetics speed movement of stability. The second phase of the thermodynamic stability (Quantum Matrix Antigravity) establishes the interaction level of strict thermodynamic stability to lowest energy between the fundamental thermodynamic force and microcrack extension stress stability (Entropy). The chemical potential of Antimicrobial cream has the enormous advantage of being a fundamental quantity, as well as temperature, deduced from the principles of thermodynamics and thus benefit from the physical and mathematical arsenal of this discipline. Also, the reactivities of silica and ortho-silicic acid and Galenic structure reactivities of the Antimicrobial skin cream (healing method) elucidate and exhibits the impact or influence of the second law of thermodynamics in the metabolism and physiology human body and all living system (human and Animal). The Antimicrobial skin cream formula are:

The final formula is: (Al,Zn,Fe₁,67MgO₃₃)Si₄O₁₀(OH)₂Na⁺Ca⁺⁺)

The Galenic structure is: Mg₃Si₄O₁₀(OH)₂(s)+Al₂Si₂O₅(OH)₄(s)+15H₂O-→6(H₄SiO₄)(s)+2Al(OH)₃+3Mg(OH)₂

During this reaction, the formation of ortho-silicic acid, aluminum hydroxide and magnesium hydroxide may occur, provided by the complete hydrolysis of Kaolin (Al₂Si₂O₅(OH)₄) and talc activated by the amount of sodium chloride, Lauryl ether sulfate, and fatty acids. In the presence of Blue Tartrazine, and vitamins. The reaction may result in a gradual solubility of Silicon and generates the ortho-silicic acid, organic silicon, aluminum hydroxide, magnesium hydroxide. The solubility of mineral silicon may generate organic silicon compound by alkylation, hydrolyze, dehydration, condensation, chelation, and cations exchanges.

The Zeta non-zero of the Antimicrobial skin cream represent the physical and chemical potential during synthesis and final process.

The Matrix thermodynamic stability first phase is occurred during osmotic and crystalline swelling synthesis step or First phase process. Therefore, hysteresis and stepwise may be observed with oxydo-Reduction activities initializing the augmentation of Cations and metalloprotein exchange adsorption providing the currents with the activities of cations and anions, which led the modification and deformation of physical Antimicrobial skin cream structures and emanate transformations topology. Obviously, the transformation, modification, and deformation emanate the curvatures transformations topology led to the passage of Montmorillonite structure through polymorphism substitution topography. During crystalline and osmotic swelling, the Antimicrobial skin cream displays multi-curvatures form on the quest of the thermodynamic stability. The fundamental thermodynamic force and microcrack extension stress stability (Entropy) exerts co-interaction and co-reaction inversely proportional to which the reactions emanate and exhibits the structure deformation with effect of kinetics stability with collisional and non-collisional particles in search of thermodynamic stability. Therefore, the kinetic stability is the consequence of the Antimicrobial cream's undulatory nature.

When the fundamental thermodynamic force regulates action or reaction, another problem arises which results from the first problem solved in the search of low stability or the condition of thermodynamic stability. We have the formations of the Collisional and non-Collisional force particles. The second phase of zeta non zero of Antimicrobial is the phase Quantum Metrix Antigravity to witch the correlation between thermodynamic and microcrack extension stress stability (Entropy) reach the strict the lowest level stability, and we have the formation of new form of potential energy, new chemical and stable components with thermodynamic lowest energy. All these processes occurs In the Antimicrobial cream synthesis process, during osmotic and crystalline swelling total charge of the Antimicrobial cream is 0.3 or 0.8, Cations offsetting the permanent and variable loads of Kaolin clay remain, for the most part, exchangeable in the environment. With the presence of Lauryl ether sulfate, blue Tartrazine, talc, mineral oil, clove oil, and vitamins the present embodiment acquires the concept of Montmorillonite function and structure. In this experience to which the Antimicrobial cream is 0.3 or 0.8 the thermodynamic law applies. The fundamental thermodynamic force Law and Entropy (The microcrack extension stress stability) are inversely proportional. Obviously, in search of thermodynamic stability, the Antimicrobial cream performs many curvature topology by kinetic and deformation thermodynamic stability we have many curvature, Reimann, Boltzmann, and natural.

The potential energy of resistance is responsible for both hysteresis and the stepwise nature. The movements of a Cations may be followed by the measurements of electrical conductivity. Also, when the cations are hydrated , the layers may be sufficiently spread apart to allow the cations to pass from one site to another and an electric current may be measured. The thermodynamic law govern universe, in the antimicrobial skin cream the current may be function of the compensating cation and the interaction between clay surface and cations. The movement of cations may follow by measurement of electrical conductivity. In the present disclosure, isomorphism, polymorphism and crystalline swelling may be observed , the static system is modeled by a balance among potential energies, attraction, and repulsion. The isomorphism substitution gives (Si4⁺→Al3⁺,Fe3⁺), in the tetrahedral site. (Al³⁺→Mg²⁺Fe²⁺ or Mg²⁺→Li²⁺). The negatively charged tetrahedral layers may be bonded by cation layers such as Mg²⁺, Al³⁺. The total energy between the particles may decreases as they approach each other. The potential energy of attraction is due to both electrostatic interaction between the interlayer cations and negative surface charge site and the vender walls between attraction site. During osmotic swelling, binary compound may be formed between two elements.

The Physicochemical Potential Topology Curvature Structure of Antimicrobial skin cream during osmotic and swelling and final phase.

The physicochemical potential topology curvature structure of the Antimicrobial skin cream exhibits two phases: The first phase(Matrix thermodynamic stability) is a phase of instability in search of thermodynamic stability during crystalline and osmotic swelling phase, so in this phase the Antimicrobial cream generates topological deformations with the presence of curvature (Normal, regular multiform, irregular multiform, Reimann, Boltzmann, Ricci curvature) in search of stability it proceeds kinetics speed movement of stability.

The second phase (Quantum Matrix Antigravity) is a phase stability and continual interaction of lowest energy. The Quantum Metrix Antigravity establishes the interaction level of strict thermodynamic stability to lowest energy between the fundamental thermodynamic force and microcrack extension stress stability (Entropy).

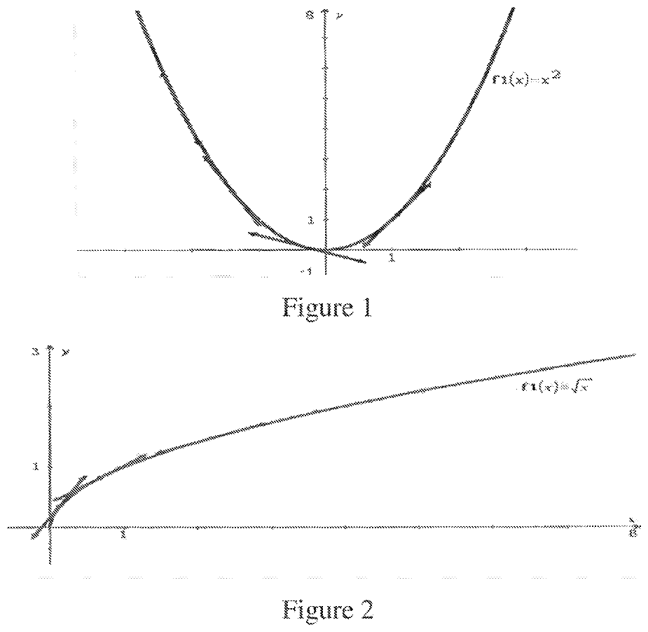

During the first phase(Matrix thermodynamic stability) The fundamental thermodynamic force and microcrack extension stress stability (Entropy) co-interact during osmotic swelling to generate and emanate irregular curvature deformation in search of thermodynamic kinetic stability. There is a relationship as shown in FIG. 1.

The co-interaction between the fundamental thermodynamic force and microcrack extension stress stability (Entropy) emanate curvature deformation topology in the search of kinetics stability during crystalline and osmotic swelling .The square function is a convex, which represent the matrix thermodynamic in search of stability.

The antimicrobial final is (Al,Zn,Fe₁,67MgO₃₃)Si₄O₁₀(OH)₂Na⁺Ca⁺⁺)

The Galenic structure is: Mg₃Si₄O₁₀(OH)₂(s)+Al₂Si₂O₅(OH)₄(s)+15H₂O-→6(H₄SiO₄)(s)+2Al(OH)₃+3Mg(OH)₂

A relationship is shown in FIG. 2. The co-interaction between the fundamental thermodynamic and macrocrack extension stress stability (Entropy) emanate curvature deformation topology in the search of kinetic stability during osmotic and crystalline swelling. The Matrix thermodynamic may be represent as follow: The square root function f(x) is a concave function on: [0,+∞], which represent The Matrix Thermodynamic in search of stability.

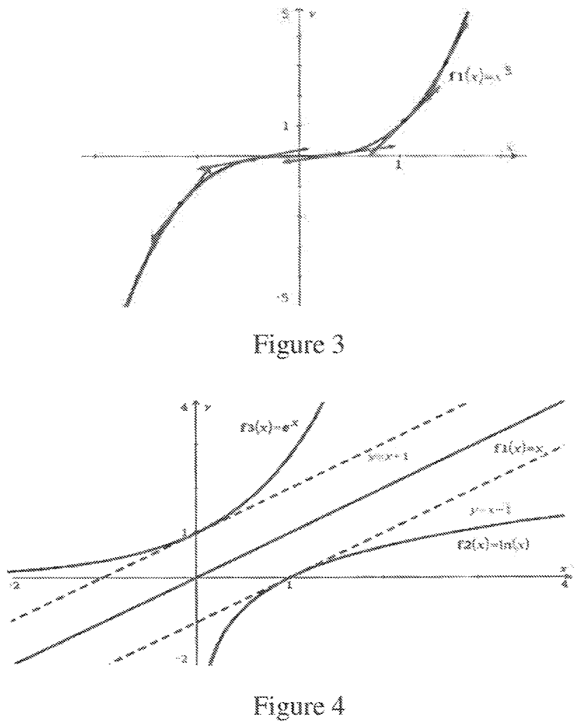

A relationship is shown in FIG. 3. The co-interaction between the fundamental thermodynamic force and microcrack extension stress stability(Entropy) emanate curvature deformation topology in the search of kinetics stability during osmotic and crystaline swelling The Matrix thermodynamic may be represented as follows: [The cube function x-→F(x)=X³ is concave on ]−∞,0[ and convex on ]0,+∞, which represent The Matrix Thermodynamic in search of stability. The Antimicrobial skin cream final formula is: (Al,Zn,Fe₁,67MgO₃₃)Si₄O₁₀(OH)₂Na⁺Ca⁺⁺).

A relationship is shown in FIG. 4.The co-interaction between the fundamental thermodynamic force and microcrack extension stress stability (Entropy) emanate curvature deformation topology in the search of kinetics stability during osmotic and crystalline swelling. The Matrix thermodynamic may be represents as follow: F1, F2, F3, which is represented by the curvature of Matrix thermodynamic in search of stability. The have the thermodynamic deformation curvature, which is represented by Reiman, Gauss, and Boltzmann. Boltzmann proposed the following equation to describe the relationship between entropy and the amount of disorder in a system.

S=klnW

In this equation, Sis the entropy of the system, k is a proportionality constant equal to the ideal gas constant divided by Avogadro's constant, In represents a logarithm to the base e, and W is the number of equivalent ways of describing the state of the system. According to this equation, the entropy of a system increases as the number of equivalent ways of describing the state of the system increases. The Entropy is always proportional to the mass particles and photon activities, electromagnetic , electrostatic force activities and levitation.

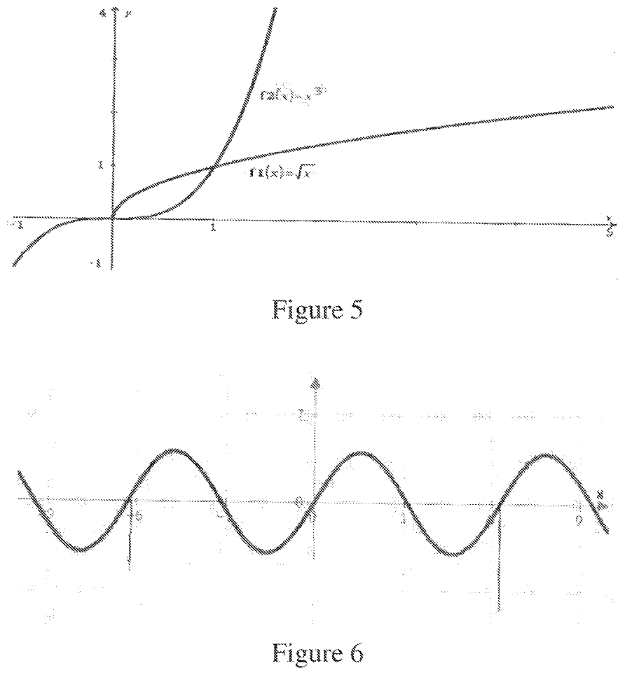

The Antimicrobial skin cream final formula is: (Al,Zn,Fe₁,67MgO₃₃)Si₄O₁₀(OH)₂Na⁺Ca⁺⁺)). A relationship is shown in FIG. 5.

The second phase of the thermodynamic strict lowest stability (Quantum Matrix Antigravity) establishes the interaction level of strict thermodynamic stability to lowest energy between the fundamental thermodynamic force and microcrack extension stress stability (Entropy).

A relationship is shown in FIG. 6. The co-interaction and co-reaction inversely proportional between the fundamental thermodynamic force and microcrack extension stress stability(Entropy) emanate curvature deformation topology of electromagnetic fields in the strict phase of lowest energy during the final synthesis phase. The Quantum Matrix Antigravity may be represented as follow: The Quantum Matrix Antigravity is a level a strict final synthesis phase. This graph curvature topology displays and shows the coordinated activity and inversely proportional of the fundamental thermodynamics force and microcrack extension stress stability (Entropy) with lowest stability with neatness, precision, and keen interaction in continual, stable level correlation, and this co-activities of the fundamental thermodynamic force and microcrack extension ability (Entropy), in the final phase of synthesis process, The fundamental thermodynamic force and microcrack extension stress stability (Entropy) govern the world, all the activities of the universe. The Antimicrobial skin cream final formula is: (Al,Zn,Fe₁,67MgO₃₃)Si₄O₁₀(OH)₂Na⁺Ca⁺⁺)

The Galenic structure is: Mg₃Si₄O₁₀(OH)₂(s)+Al₂Si₂O₅(OH)₄(s)+15H₂O-→6(H₄SiO₄)(s)+2Al(OH)₃+3Mg(OH)₂

Thermodynamics and Entropy are inversely proportional. Moreover, the statistics which react to the spacings between the different zero of Reiman's function are exactly the same as the statistics which govern the energy levels of the theoretical random atoms. If we build an atom with random bricks we measure energy levels we will find the statistics of the state of Reiman. We have the connection and correlation between quantum mechanics and prime numbers. The differences and between the zero of Reiman are the same as the statistics of the energy levels which exist at the level of the random atoms. The fundamental thermodynamic force governs universe

The Antimicrobial final formula is:

(Al,Zn,Fe₁,67MgO₃₃)Si₄O₁₀(OH)₂Na⁺Ca⁺⁺)

Riemann's Hypothesis Zeta function

$$ζ ( s ) = {\sum }_{n = 1}^{∞} \frac{a}{{n}^{s}} = \frac{1}{{1}^{s}} + \frac{1}{{2}^{s}} + \frac{1}{{3}^{s}} + …$$

Leonhard Euler introduces it (without giving him a name) only for real values of the argument (but also for s=1), in stability structure topology of Reiman curvature

$$ζ ( s ) = {\prod }_{p prime} \frac{1}{1 - {p}^{- s}} = \frac{1}{1 - {2}^{- s}} * \frac{1}{1 - {3}^{- s}} * \frac{1}{1 - {5}^{- s}} * \frac{1}{1 - {7}^{- s}} * \frac{1}{1 - {11}^{- s}}$$

The structure and the geometric design curvature of the cream Antimicrobial cream performs and obeys the structure of the differential geometry with possible structures most often metric Reimanienne. During crystalline and osmotic swelling phase, on the quest of thermodynamic stability. The silicon performs rotates and performs many physical transformations with many curvature led to the transformation Kaolinite structure to Montmorillonite. We have: First, silicon may be present in independent silicates, wherein no oxygen atom is bonded to two silicon atoms. The SiO₄ tetrahedral anions are associated with cations like Mg₂SiO₄, where a hydrogen bond is present between ions and a Van der Waals bond is present at the top of each silicate.

Second, silicon may be present in silicates with finite tetrahedral groups, wherein the tetrahedral of the same group may be combined in the minerals such that an oxygen atom is a common sum with two Si₂0₇. Thus, there may be a hydrogen bond between a cation and inorganic colloid and a Van der Waals bond at the top of each silicate, wherein the top of the silicate is defined as the interlayer connection by Van der Waals attractions.

Third, the silicon may be present in silicates arranged in chains, wherein the tetrahedral combine to form an infinite linear chain, and the corresponding silicates may have a fibrous texture. When the chain is a simple chain, its chemical composition is SiO₃, and the properties are similar to the tetrahedral anion SiO₂(OH). This arrangement may include hydrous sodium silicate with a hydrogen bond between colloids and a Van der Waals bond at the top of each silicate.

Fourth, the silicon may be present in a lamellar silicate, wherein the tetrahedron chains pool some of the vertices to form planar networks. Thus, two chains of juxtaposed pyroxenes, of link SiO3, provide the double chain of the amphiboles Si₄O₁₁. If the chain continues so that three atoms of each of the tetrahedral are common to three other tetrahedral, a planar array of tetrahedrons bound by three of their vertices whose chemical composition is Si205 is formed.

All of the above structural type of silicates may be rotated and organized during the synthesis process of the cream of the present disclosure due to the swelling process caused by the cation imbalance created with the presence of Brilliant Blue, fatty acids, and lauryl ether sulfate. The structures and ions present in the Cream. The fundamental thermodynamics force and microcrack extension govern the universe. Thermodynamics and Entropy are inversely proportional. Moreover, the statistics which react to the spacings between the different zero of Reiman's function are exactly the same as the statistics which govern the energy levels of the theoretical random atoms. If we build an atom with random bricks we measure energy levels we will find the statistics of the state of Reiman. We have the connection and correlation between quantum mechanics and prime numbers. The differences and between the zero of Reiman are the same as the statistics of the energy levels which exist at the level of the random atoms. In the conditions of co-interaction and inversely proportional of the fundamental thermodynamic and microcrack extension stress (entropy) in a constant and permanent regulation giving electromagnetic fields. The swell as the water gain entry into the lattice structure and shrink if the water, Lauryl ether sulfate, and fatty acid are removed. The total energy between the particles decrease as they approach each other, there is attraction between them which, case the particles approach each other and flocculate essentially to face configuration or edge to edge. When the cations are hydrated, the layers are sufficiently spaced to allow the cations to pass from one site to another and an electric current is measured. This current is obviously a function of a compensating cations and the interaction between clay the clay surface des cations. However, there is also an electric current in the dry state which generally ascribed to the compensating cation. The Antimicrobial cream final formula is: (Al,Zn,Fe₁,67MgO₃₃)Si₄O₁₀(OH)₂Na⁺Ca⁺⁺)

Normal curvature in the Antimicrobial skin cream

An exemplary of normal curvature with Theorem of Euler for the curvature

We have the function

Tₚ¹S→IR

X→Kx

When the function reaches its extrema in the eigenvectors (units) of the Weingarten operator Wp. If X1 is such an eigenvector associated with the eigenvalue M and if X makes an angle with X1 then we have:

kX=KX1 cos(2 θ)+KX2 sin(2 θ)

where X2 is a unit eigenvector associated the eigenvalue λ2. Therefore:

λ1=kX1+λ2=kX2.

Demonstration

By definition kX 32 11p(X,X) 32 (h−dnp(X),X)

The vector X being unitary

We have:

X=cosθX1+sinθ×2.

On a −dnp(X)=cos.θλ1×1+sin θλ2X2

And kX=cos2θ.λ1+sin2θ.λ12

Corollary
Let Xθ=cosθ.X1+sinOθX2.

The average of the function 0→kXθ is the mean curvature:

$$\frac{1}{2 π} {\int }_{0}^{2 π} K X o d θ = H ( P )$$

### Demonstration.

This is an immediate calculation that use Euler formula for curvature

$$\frac{1}{2 π} {\int }_{0}^{2 π} K X o d θ = H ( P ) = \frac{1}{2 π} {\int }_{0}^{2 π} ( {cos}^{2} X θ . λ 1 + {sin}^{2} X θ ) . λ 1 d θ = \frac{λ 1 + λ 2}{2}$$

The fundamental original universe thermodynamic energy force govern universe. Thermodynamic energy and Entropy are inversely proportional. We may have in the Antimicrobial cream asymptotic curvature an curvature lineshaving

A point PϵS which λ1(p)=λ2(p) is called an umbilical point. All points of the sphere or the plane are umbilical

Let f:U→S be a regular parametric surface, injective and without a point.

y: I→U

∀t→(u(t),v(t)) a regular curve. Then y=f°y is a line of curvature if and only if:

$$d e T ( \begin{matrix}( {v}^{′} ) 2 & - {u}^{′} {v}^{′} & ( {u}^{′} ) 2 \\ E & f & g \\ c & m & n\end{matrix} ) = 0$$

Demonstration. Since f has no umbilical point, two principal curvature functions λ, λ2:U→R can be unambiguously defined. The parameterized curve y is a line of curvature if there are i=1 or 2 such that

t∈l, dn(γ(t))=λiγ(t) is the gamma t vector in the base (fu, fv) the previous equality is written:

$$\frac{1}{E G} - {F}^{2} ( \begin{matrix}GL - F M & GM - FN \\ EM - FL & EN - F M\end{matrix} ) ( \begin{matrix}{U}^{′} \\ {V}^{′}\end{matrix} )$$

Hence, multiplying the first line by V′ and the second by v′

$$\frac{G L - F M}{E G - {F}^{2}} ( {U}^{′} {V}^{′} ) + \frac{G M - F N}{E G - {F}^{2}} {( {V}^{′} )}^{2} = {λ}_{i} ( {U}^{′} {V}^{′} ) \\ \frac{G L - F M}{E G - {F}^{2}} {( {U}^{′} )}^{2} + \frac{G M - F N}{E G - {F}^{2}} ( {U}^{′} {V}^{′} ) = {λ}_{i} ( {U}^{′} {V}^{′} )$$

By matching, we obtain an expression which is, by a factor, the determining factor in the lemma. Corollary. If F≡0 and M≡0 then u7→f(u, vo) and v7→>f (uₒ, v) are lines of curvature.

We may also have a geodesic curvature:

Let U→IR³, for i=1 or 2, on two parametric surfaces of class C² isometric and let λ: I→U. If λ1=fᵢ Cy is a geodesic of S₁=f₁(U) then λ_{2(VECTOR)}=f₂°λ a geodesic of S₂=f₂(U)

The fundamental universe origin force thermodynamic energy force govern universe. The thermodynamic and Entropy are inversely proportional. In the universe as in the Antimicrobial cream we found the same parameters law (The fundamental force Thermodynamic force and Entropy). Therefore, we have same properties. The level state of a system in thermodynamic equilibrium is determined by the values of its intensive parameters, such as pressure or temperature. More specifically, thermodynamic equilibrium is characterized by the minimum of a thermodynamic potential, such as Helmholtz free energy for constant temperature and volume systems, or Gibbs free enthalpy. The chemical reactions in the Antimicrobial cream as in the universe manifest and occur with the lowest energy to ensure the thermodynamic stability

The Zeta Zero- or zero-point energy of the Antimicrobial skin cream during the synthesis process

The zeta zero-point energy of the Antimicrobial skin cream is related to the reactivity of Kaolin structure on the composition, which contains inorganic colloid negative and metalloproteins (Cation) provided by the presence of water in the presence gelatin, vitamins, and Blue Tartrazine, and performing oxydo-reduction hysteresis and stepwise cation adsorption and rehabilitation of skin in the process of wound healing. When the total charge and energy of cream equal zero or high level strict stability thermodynamic, the clay kaolin load on the Antimicrobial cream is too high. However, there is no transformation, no water absorption, no swelling effect, no oxydo-reduction stepwise and hysteresis, we don't have any

exchange the surroundings, we reach the equilibrium state. The thermodynamic is reaching stability. The entropy equal zero. In this point the Antimicrobial skin reaches the strict thermodynamic stability the Antimicrobial system energy is zero. This stability obeys to the 3rd thermodynamic law.

$${lim}_{T \to 0} {( Δ S )}_{T} = 0$$

The determination of the overall charge is made by measuring the zeta potential. The third principle of thermodynamics is associated with the descent to its fundamental quantum state of a system whose temperature approaches a limit which defines the notion of absolute zero. Also known as Nernst's principle, the third principle of thermodynamics is: Third principle of thermodynamics At the limit of absolute zero, a pure body in a perfect crystalline state has zero entropy.

$${( \frac{\partial E}{\partial S} )}_{V , {n}_{i}} = 0$$

at T=0K, ∀p, S=0

The reasons and the origins of the virtues functions of the effectiveness of the cream skin Antimicrobial. The reasons that justify the functional effectiveness of the cream are multiple, the most important are:

1) Kaolin clay has a great use in the pharmaceutical industry

2) The abundance of mineral elements in the universe and for the functioning of the human body;

3) the cream contains essential and important elements of the creation of life on earth

1) The therapeutic power and virtues of clay justifies its intimate correlation with the human body metabolism functioning. Therefore, beneficial effects for human health of clay minerals, describing their use in pharmaceutical formulations, spas and aesthetic medicine. Their therapeutic action as active principles in pharmaceutical formulations orally administered (gastrointestinal protectors, laxatives, antidiarrhoeics) or for topical applications (dermatological protectors and cosmetics) is described. Their use as excipients and their influence in the bioavailability of the organic active principle is also described, both in the liberation process and in its possible degradation effect. Among their uses in spas, clay minerals therapeutic activity, in geotherapy, pelotherapy and paramuds is commented upon. Moreover, the applications of the clay minerals in aesthetic medicine (to clean and moisturize the skin and to combat compact lipodystrophies, acne and cellulite) are also described. All these Therapeutic virtues justifies the correlation and similarities of the Antimicrobial skin cream in the process of healing skin disease and wound healing under supervision of the fundamental thermodynamic force and microcrack extension stress stability to which the second thermodynamic law reactivities performs and supervise the physiologic reactions to facilitate metabolism in the human body with presence of mineral element and Silicon reactivities.

2) There is an abundance of Antimicrobial skin cream's mineral element in the universe. The great character and efficiency of the cream can be verified by the correlation link function of the human body and universe. The most common metalloprotein or mineral element fund in the Antimicrobial cream are present in human body and universe and interstellar dust. Also, all planets contains the different elements generated and formed in the infinitely large or macroscopic scale (Na, Ca, Si, O, H, C, Mg, Zn, Fe), the same chemical are also found the human body or living system (plant, animal),and microscopic scale as well as the Antimicrobial skin cream. The fundamental thermodynamic law force govern universe. This correlation extends to a great intimacy between the infinitely small and the infinitely large. Also, in a relationship that verifies rational and scientific way that living systems (humans, animals, plants) are dust from stars. The thermodynamic law influences the metabolism reactivities of all universe entity as well as human by performing, and creating physiological Entropy to regulate human functioning metabolism and heal skin pathology with presence of Silicon and ortho-silicic acid. The Kaolin formula is

(Ca,Na,H)(Al,Mg,Fe,Zn)₂(Si,Al)4O₁₀(OH)₂*H₂O.

The Antimicrobial skin cream formula is (Al,Zn,Fe₁,67MgO₃₃)Si₄O₁₀(OH)₂Na⁺Ca⁺⁺).

By taking account of thermodynamic law reactivities and its influence in the universe specially the thermodynamic level, which is continuously inversely proportional in relation with the ortho-silicic reactivities, creating physiological Entropy. The silicon contained in clay and the Antimicrobial skin cream creates the Physiological Entropy to boost the human immune system, induce immune response, improve cellular communication and help the human body in the immune process of defending the human body against skin disease. This process is effective with the benefit of the reactivity of ortho-silicic acid.

3) The Antimicrobial skin cream composition synergies in origin of Living Cell, DNA, and Life(Genetic Takeover).

Genetic Takeover is about the origins of life on earth. Obviously, in the earth early time. The fundamental force thermodynamic law in the control of silicon, ortho-silicic and clay reactivities interacted with the entropy (microcrack stress stability) led the chemical evolution which creates a ‘primeval soup’ that leads to the formation of prebiotic elementary. Obviously, the primeval soup was clay in composition with inorganic colloids, silicon, ortho-silicic acid, and mineral element; (Na⁺, Ca²⁺, K⁺, Mg²⁺, Zn²⁺, Al³⁺, Fe³⁺, Fe²⁺, Cu⁺, Cu²⁺, Ti⁴⁺) in sea water (in composition with sodium chloride, mineral elements, The ortho-silicic acid and silicon organic of clay mineral such as Na⁺, Ca²⁺, K⁺, Mg²⁺, Zn²⁺, Al³⁺, Fe³⁺, Cu⁺, Cu²⁺, and Ti⁴⁺. The ortho-silicic acid and silicon organic of clay mineral, simulated ancient seawater, clay, silicon organic and ortho-silicic forms a hydrogel, which performed Oxydo-Reduction of mineral elements most (Iron, cooper) with hysteresis, stepwise activities creating mass of microscopic spaces capable of soaking up liquids like a sponge. Over billions of years, chemicals confined in those spaces could have carried out the complex reactions, the supra-molecular assemblies, addition, transformation, and resonances (complex mesomeres or resonance reactions between cations mineral and colloids or negative complex) that giving many pre-biotic compound and formed afterwards, proteins, DNA, and probiotics in the presence light and Sun energies, and eventually all the machinery that makes a living cell work. Clay that contained ortho-silicic acid and silicon organic hydrogels could have confined and protected those chemical processes until the membrane that surrounds living cells developed. to illustrate The ferruginous strata contain fossils of primitive lives shaped by old bacteria (stromatolites).

S_{sys}>0 implies that the system becomes more disordered during the reaction.

S_{sys}<0 implies that the system becomes less disordered during the reaction. The Kaolin formula is (Ca,Na,H)(Al,Mg,Fe,Zn)₂(Si,Al)4O₁₀(OH)₂*H₂O. The Antimicrobial skin cream formula is (Al,Zn,Fe₁,67MgO₃₃)Si₄O₁₀(OH)₂Na⁺Ca⁺⁺).

The benefits of the physicochemical potential characteristic activities of the cream Antimicrobial

The physicochemical potential characteristic may perform many benefices in our lives, The most important are,

first the identification, quantification, and determination of thermodynamic instability and thermodynamic stability by analyzing the physical aspect of deformation curvature (Matrix thermodynamic and Quantum Metrix Antigravity) emanated by the co-interaction and co-reaction between the fundamental thermodynamic force microcrack extension stress stability. The physical aspect curvature may be related to magnitude, frequency and electromagnetic field.

Second, the systematic use of the virtues of the chemical, biochemical and therapeutic properties of the antimicrobial cream active ingredient (Ortho-silicic acid) to produce pharmaceutic products(Antimicrobial Baby cream). The Antimicrobial Baby cream formula is: The Antimicrobial baby skin may be synthetized with two reactions. The ortho-silicic and Magnesium Hydroxide cream acid by talc hydrolyze in the presence of vitamin. We have:

I) 2Mg₃SiO₄(OH)₂+6H₂O--->SiO₂+H₄SiO₄+6Mg(OH)₂ PH=7.4

We may also, synthetize the ortho-silicic and Magnesium hydroxide by hydrolyze Talc in the presence of small amount of ortho-silicic acid we have: II) 2Mg₃SiO₄(OH)₂+H₂O₄SiO₄6H₂O--->SiO₂₊₂H₄SiO₄+6Mg(OH)₂

Determination, identification and quantification of Thermodynamic aspect stability. The physicochemical potential activities of the Antimicrobial cream make it possible to detect the amplitudes of the activities of co-interaction and co-reaction of the fundamental thermodynamic force and microcrack extension stress stability (Entropy) which is translated and justified by deformations of curvatures considered irregular and unstable then deformations of regular curvatures very strict thermodynamically stable. The physicochemical potential makes it possible to identify instability and strict stability of the universe fundamental thermodynamic force. The fundamental thermodynamic force governs universe, and the fundamental thermodynamic and microcrack extension stress stability (Entropy) are inversely proportional. The Quantum Metrix Antigravity is the strict, net, and keen regulation of co-interaction and co-reactivities between the fundamental thermodynamic and microcrack extension stress stability (Entropy) established during the synthesis final phase of Antimicrobial skin cream in the search of co-reactivities of lowest energy possible to conform to the condition of thermodynamic strict stability. In correlation of the concept of microscopic and macroscopic scale by mirror effect of the Antimicrobial skin cream. The physicochemical potential or electromagnetic co-interaction between the fundamental thermodynamic force and Entropy, help to identify, detect, and classify the thermodynamic force stability by frequency, and amplitude emanated by the deformation of universe space. The physicochemical potential help to characterize and identify deformation in microscopic and macroscopic scale. The physicochemical help to distinguish by the magnitude and frequency the deformation curvature of gravitational wave, X-ray, gamma ray, Radio, infrared, UV. The structure and the geometric design of the differential geometry with possible structures most often metric Reimanienne. The Antimicrobial skin cream formula final formua is: (Al,Zn,Fe₁,67MgO₃₃)Si₄O₁₀(OH)₂Na⁺Ca⁺⁺).

The galenic Antimicrobial cream formula is Mg₃Si₄O₁₀(OH)₂(s)+Al₂Si₂O₅(OH)₄(s)+15H₂O−→6(H₄SiO₄)(s)+2Al(0H)₃+3Mg(OH)₂

The Ortho-silicic acid active ingredients reactivity serves reference to Produce the Baby Antimicrobial cream. In the following detailed description of the invention (The Antimicrobial baby cream), numerous details, examples, and embodiments of the invention are described. However, it will be clear and apparent to one skilled in the art that the invention is not limited to the embodiments set forth and that the invention can be adapted for any of several applications.

The cream of the present disclosure may be used to heal and rejuvenate the skin and may comprise the following elements. This list of possible constituent elements is intended to be exemplary only, and it is not intended that this list be used to limit the device of the present application to just these elements. Persons having ordinary skill in the art relevant to the present disclosure may understand there to be equivalent elements that may be substituted within the present disclosure without changing the essential function or operation of the device.

| | |
|---|---|
| 1. | Talc |
| 2. | Water |
| 3. | Glycerin |
| 4. | Sodium lauryl ether sulfate Menthol |
| 5. | Sodium chloride |
| 6. | Gelatin |
| 7. | Vitamins |
| 8. | Ortho-silicic acid powder |
| 8. | Sodium Benzoate |
| 9. | Sodium salicylate |
| 10. | Mineral Oils |
| 11. | propylene glycol |
| 12. | Isopropyl palmitate |
| 13. | Lanolin |
| 14. | Parfum |

The various elements of the cream of the present disclosure may be related in the following exemplary fashion. It is not intended to limit the scope or nature of the relationships between the various elements and the following examples are presented as illustrative examples only.

By way of example, The cream of the present invention with ortho-silicic acid powder is designed to soothe and help protect the dry, sensitive skin of a young child. The cream is the solution to protect babies against chronic lung irritation, and asthma to reduce rushes and diaper and diaper irritation in babies and infant. The antimicrobial cream is a healing ointment advanced therapy to help heal Diaper rash chapped skin. The ortho-silicic acid is the main ingredient active of the Antimicrobial baby cream.

The Antimicrobial baby skin may be synthetized with two reactions. The ortho-silicic and Magnesium Hydroxide cream acid by talc hydrolyze in the presence of vitamin.

2Mg₃SiO₄(OH)₂+6H₂O→SiO₂+H₄SiO₄+6Mg(OH)₂

The formation of Si(OH)4 is shown further below:

(a) Hydrolyze Si4+ with water dissociation:

Si⁴⁺+H₂O→Si(OH)³⁺+H⁺

Si(OH)³⁺+H₂O→Si(OH)₂²⁺+H⁺

Si(OH)₂²⁺+H₂O→Si(OH)₃⁺+H⁺

Si(OH)₃⁺+H²O→Si(OH)₄+H⁺

(b) Thus, the result is:

Si⁴⁺+4H₂O→Si(OH)₄+4H³⁰

H⁺+OH⁻→H2O (1), wherein the pH=7.4

One may also synthetize the ortho-silicic and Magnesium hydroxide by hydrolyzing talc in the presence of small amount of ortho-silicic acid completing the following reaction:

2Mg₃SiO₄(OH)₂+6H₂O+H₄SiO₄→SiO₂+2H₄SiO₄+6Mg(OH)₂

Both of the reactions above had a pH of 7.4.

The addition of talc may provide the additional silicon and magnesium. As mentioned, the Si—O bond may be under mechanical stress. And, if it can be broken, a microcrack in the silicate may occur. Water molecules in the vicinity of the stress point will be attracted to the Si—O bond so that its oxygen bonds to the silicon via the oxygen lone pairs. Also, one of its hydrogens can hydrogen bond to the bridging oxygen. Next, there is a simultaneous transfer of protons and electrons. Thereafter, the new bonds are formed—one between oxygen and silicon and the others between hydrogen and oxygen. Finally, the original bond between oxygen and silicon is destroyed. The final step is breakage of the hydrogen bond and the transferred hydrogen to give an Si—OH on each fractured surface, leaving multiple H4SiO4 (s) compounds. Thus, the combination of a weakened bon and constant stress created by The Matrix thermodynamic and The Quantum Metrix Antigravity we have:

We have : (SiO2)ₙ+2H₂O↔(SiO2)ₙ₋₁+Si(OH)₄, wherein n>1.

Sodium Salicylates are derivatives of salicylic acid that occur naturally in plants and serve as a natural immune hormone and preservative protecting the plants against diseases, insects, fungi, and harmful bacteria. Salicylates can also be found in many medications, perfumes and preservatives. Glycerin is a moisturizer to treat or prevent dry, rough, scaly, itchy skin and minor skin irritations. Propylene glycol is also as moisturizer in cosmetic products and as a dispersant in fragrances. Isopropyl palmitates or Ethylhexyl Palmitate, also called Octyl Palmitate act as lubricants the skin's surface, which give the skin soft and smooth appearance. Lanolin is used as a moisturizer to treat or prevent dry, rough, scaly, itchy skin and minor skin irritations (e.g., diaper rash, skin burns from radiation therapy). Emollients are substances that soften and moisturize the skin and decrease itching and flaking. Some products (e.g., zinc oxide, white petrolatum) are used mostly to protect the skin against irritation (e.g., from wetness).

### EXAMPLE 1

### Talc Powder Without Ortho-Silicic Acid Powder

The following ingredients are mixed in a batch: about 100 kg talc; about at least 200 kg but no more than 250 kg lanolin; about 103 kg petroleum jelly; about 1 kg sodium lauryl ether sulfate; about 100 g sodium chloride; about 50 g sodium benzoate; about 5 liters mineral oil; about 7 liters propylene glycol; about 1 liter sodium salicylate solution concentrated at 48.5% by volume; about 7 liters water; and about 100 g gelatin.

In a first container, 100 kg of talc is mixed with 200 kg of lanolin and 103 kg of petroleum jelly until a homogeneous cream paw is obtained. In a second container number, 1 kg of Lauryl ether sulfate, 100 g of sodium chloride, 7 L of water, 1L of solution of sodium salicylate (48.5% by weight) are mixed.

After complete homogenization, 7 L of Isopropyl palmitate, 7 L of propylene glycol, ½ L of Glycerin (optional), 5 L of mineral oil, 50 g of sodium benzoate are added and further mixed until the formation of a colloidal solution recipient number Then, the homogeneous solution of the first container is mixed with the solution in the second container, and then homogenized with a mixer.

### EXAMPLE 2

### Talc Powder with Ortho-Silicic Acid Powder

The following ingredients are mixed in a batch: about 50 kg talc; about 50 kg ortho-silicic acid powder; about at least 200 kg but no more than 250 kg lanolin; about 103 kg petroleum jelly; about 1 kg sodium lauryl ether sulfate; about 100 g sodium chloride; about 50 g sodium benzoate; about 5 liters mineral oil; about 7 liters propylene glycol; about 1 liter sodium salicylate solution concentrated at 48.5% by volume; about 7 liters water; and about 100 g gelatin.

In a first container, 100 kg of talc is mixed with 200 kg of lanolin and 103 kg of petroleum jelly until a homogeneous cream paw is obtained. In a second container number, 1 kg of Lauryl ether sulfate, 100 g of sodium chloride, 7 L of water, 1L of solution of sodium salicylate (48.5% by weight) are mixed.

After complete homogenization, 7 L of Isopropyl palmitate, 7L of propylene glycol, ½ L of Glycerin (optional), 5 L of mineral oil, 50 g of sodium benzoate are added and further mixed until the formation of a colloidal solution recipient number Then, the homogeneous solution of the first container is mixed with the solution in the second container, and then homogenized with a mixer.

Persons of ordinary skill in the art may appreciate that numerous design configurations may be possible to enjoy the functional benefits of the inventive systems. Thus, given the wide variety of configurations and arrangements of embodiments of the present invention the scope of the invention is reflected by the breadth of the claims below rather than narrowed by the embodiments described above.

### INDUSTRIAL APPLICABILITY

Embodiments of the disclosed invention are useful for treating microbial skin infections.

What is claimed is:

1. A cream for treating the skin, the cream comprising:
talc;
lanolin;
petroleum jelly sodium lauryl ether sulfate; CH3(CH2)11(OCH2CH2)nOSO3Na, where n is from about 2 to about 3;
sodium chloride;
water;
sodium salicylate;
Lauryl ether sulfate;
Isopropyl palmitate;
propylene glycol;
mineral oil; and
sodium benzoate.

2. The cream of claim 1, further comprising glycerin.

3. The cream of claim 2, further comprising gelatin.

4. The cream of claim 3, further comprising a mixture of vitamins.

5. The cream of claim 4, wherein the mixture of vitamins comprises at least two members of the vitamin set consisting of vitamin A, vitamin E, vitamin D, vitamin C, vitamin B2, vitamin B5, vitamin H, vitamin B6, and vitamin D.

6. The cream of claim 5, wherein a batch of the cream comprises:
about 100 kg talc;
about at least 200 kg but no more than 250 kg lanolin;
about 103 kg petroleum jelly;
about 1 kg sodium lauryl ether sulfate;
about 100 g sodium chloride;
about 50 g sodium benzoate;
about 5 liters mineral oil;
about 7 liters propylene glycol;
about 1 liter sodium salicylate solution concentrated at 48.5% by volume;
about 7 liters water; and
about 100 g gelatin.

7. The cream of claim 5, further comprising ortho-silicic acid powder.

8. The cream of claim 7, wherein a batch of the cream comprises:
about 50 kg talc;
about 50 kg ortho-silicic acid powder;
about at least 200 kg but no more than 250 kg lanolin;
about 103 kg petroleum jelly;
about lkg sodium lauryl ether sulfate;
about 100 g sodium chloride;
about 50 g sodium benzoate;
about 5 liters mineral oil;
about 7 liters propylene glycol;
about 1 liter sodium salicylate solution concentrated at 48.5% by volume;
about 7 liters water; and
about 100 g gelatin.

9. The cream of claim 1, wherein the cream has a pH of about 7.4.
